(19) United States
(12) Patent Application Publication
(10) Pub. No.: US 2023/0346332 A1
(43) Pub. Date: Nov. 2, 2023

(54) IMAGING METHODS USING MULTIPLE RADIATION BEAMS

(71) Applicant: SHENZHEN XPECTVISION TECHNOLOGY CO., LTD., Shenzhen (CN)

(72) Inventors: Peiyan CAO, Shenzhen (CN); Yurun LIU, Shenzhen (CN)

(21) Appl. No.: 18/211,777

(22) Filed: Jun. 20, 2023

Related U.S. Application Data
(63) Continuation of application No. PCT/CN2021/070272, filed on Jan. 5, 2021.

(51) Int. Cl. A61B 6/00

(52) U.S. Cl. CPC A61B 6/5241; A61B 6/40; A61B 6/4233

(57) ABSTRACT

Disclosed herein is a method, comprising: sending one by one M radiation beams (radiation beams (i), i=1, . . . , M) toward a same scene, M being an integer greater than 1; for i=1, . . . , M, capturing with a same image sensor a partial image (i) of the scene using radiation of the radiation beam (i) after the radiation of the radiation beam (i) passes through the scene; and stitching the partial images (i), i=1, . . . , M of the scene resulting in a stitched image of the scene, wherein said stitching is based on relative positions of the M radiation beams with respect to each other.

### BACKGROUND

A radiation detector is a device that measures a property of a radiation. Examples of the property may include a spatial distribution of the intensity, phase, and polarization of the radiation. The radiation may be one that has interacted with an object. For example, the radiation measured by the radiation detector may be a radiation that has penetrated the object. The radiation may be an electromagnetic radiation such as infrared light, visible light, ultraviolet light, X-ray, or y-ray. The radiation may be of other types such as a-rays and (3-rays. An imaging system may include an image sensor having multiple radiation detectors.

### SUMMARY

Disclosed herein is a method, comprising sending one by one M radiation beams (radiation beams (i), i=1, . . . , M) toward a same scene, M being an integer greater than 1; for i=1, . . . , M, capturing with a same image sensor a partial image (i) of the scene using radiation of the radiation beam (i) after the radiation of the radiation beam (i) passes through the scene; and stitching the partial images (i), i=1, . . . , M of the scene resulting in a stitched image of the scene, wherein said stitching is based on relative positions of the M radiation beams with respect to each other.

In an aspect, for i=1, . . . , M−1, the radiation beam (i) and the radiation beam (i+1) share some radiation particle paths.

In an aspect, for i=1, . . . , M, the radiation beam (i) after passing through the scene falls entirely within an active area of the image sensor.

In an aspect, for i=1, . . . , M, the radiation beam (i) after passing through the scene falls entirely within a same active area of the image sensor.

In an aspect, for i=1, . . . , M, the image sensor is at a position (i) with respect to the scene when the image sensor captures the partial image (i) of the scene, and said stitching is not based on the positions (i), i=1, . . . , M of the image sensor with respect to each other.

In an aspect, said stitching the partial images (i), i=1, . . . , M comprises: for i=1, . . . , M, determining a signal area (i) of the partial image (i); and aligning the signal areas (i), i=1, . . . , M resulting in the stitched image of the scene, wherein said aligning is based on the relative positions of the M radiation beams with respect to each other.

In an aspect, said determining the signal area (i) comprises determining multiple picture elements of the partial image (i) on a signal area border line (i) of the signal area (i).

In an aspect, the signal area border line (i) has a shape of a rectangle.

In an aspect, said sending one by one the M radiation beams comprises moving a mask between a radiation source and the scene as the image sensor captures the partial images (i), i=1, . . . , M, and the mask comprises a mask window such that radiation from the radiation source that passes through the mask window results in the M radiation beams.

In an aspect, said capturing with the same image sensor comprises moving the image sensor with respect to the scene as the image sensor captures the partial images (i), i=1, . . . , M.

Disclosed herein is an imaging system, comprising a radiation beam generator configured to generate one by one M radiation beams (radiation beams (i), i=1, . . . , M) toward a same scene, M being an integer greater than 1; and an image sensor configured to (A) for (i), i=1, . . . , M, capture a partial image (i) of the scene using radiation of the radiation beam (i) after the radiation of the radiation beam (i) passes through the scene, and (B) stitch the partial images (i), i=1, . . . , M of the scene resulting in a stitched image of the scene based on relative positions of the M radiation beams with respect to each other.

In an aspect, for i=1, . . . , M−1, the radiation beam (i) and the radiation beam (i+1) share some radiation particle paths.

In an aspect, for i=1, . . . , M, the radiation beam (i) after passing through the scene falls entirely within an active area of the image sensor.

In an aspect, for i=1, . . . , M, the radiation beam (i) after passing through the scene falls entirely within a same active area of the image sensor.

In an aspect, for i=1, . . . , M, the image sensor is configured to be at a position (i) with respect to the scene when the image sensor captures the partial image (i) of the scene, and said stitching of the partial images (i), i=1, . . . , M is not based on the positions (i), i=1, . . . , M of the image sensor with respect to each other.

In an aspect, the image sensor is configured to stitch the partial images (i), i=1, . . . , M of the scene by: for i=1, . . . , M, determining a signal area (i) of the partial image (i), and aligning the signal areas (i), i=1, . . . , M resulting in the stitched image of the scene, wherein said aligning is based on the relative positions of the M radiation beams with respect to each other.

In an aspect, said determining the signal area (i) comprises determining multiple picture elements of the partial image (i) on a signal area border line (i) of the signal area (i).

In an aspect, the signal area border line (i) has a shape of a rectangle.

In an aspect, the radiation beam generator comprises a radiation source and a mask which comprises a mask window, and the mask is configured to move and allow some radiation from the radiation source to pass through the mask window resulting in the radiation beams (i), i=1, . . . , M.

In an aspect, the image sensor is configured to move with respect to the scene as the image sensor captures the partial images (i), i=1, . . . , M.

### BRIEF DESCRIPTION OF FIGURES

FIG. 1 schematically shows a radiation detector, according to an embodiment.

FIG. 2A schematically shows a simplified cross-sectional view of the radiation detector, according to an embodiment.

FIG. 2B schematically shows a detailed cross-sectional view of the radiation detector, according to an embodiment.

FIG. 2C schematically shows a detailed cross-sectional view of the radiation detector, according to an alternative embodiment.

FIG. 3 schematically shows a top view of a package including the radiation detector and a printed circuit board (PCB), according to an embodiment.

FIG. 4 schematically shows a cross-sectional view of an image sensor including a plurality of the packages of FIG. 3 mounted to a system PCB (printed circuit board), according to an embodiment.

FIG. 5A-FIG. 5E illustrate an imaging session, according to an embodiment.

FIG. 6 shows a flowchart summarizing and generalizing the imaging session.

FIG. 7 shows the radiation beams used in the imaging session, according to an embodiment.

### DETAILED DESCRIPTION

Radiation Detector

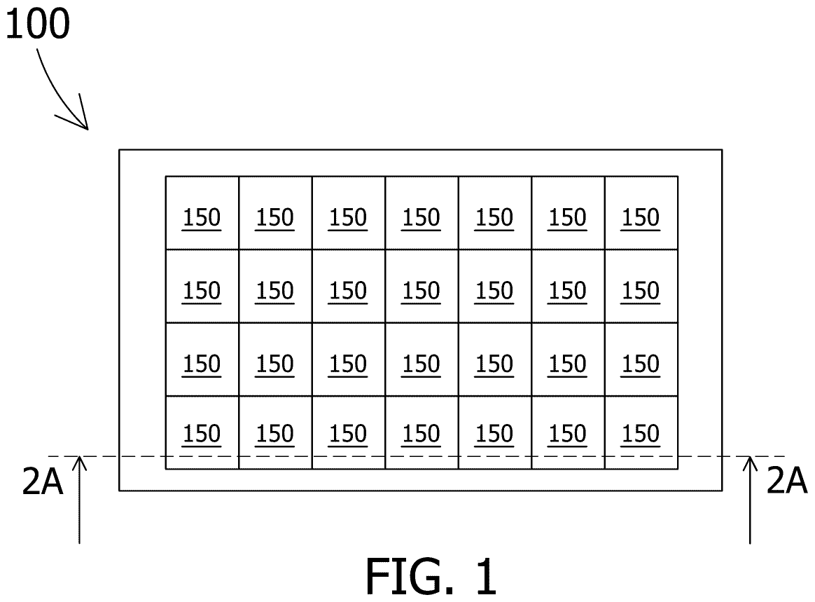

FIG. 1 schematically shows a radiation detector 100, as an example. The radiation detector 100 may include an array of pixels 150 (also referred to as sensing elements 150). The array may be a rectangular array (as shown in FIG. 1), a honeycomb array, a hexagonal array, or any other suitable array. The array of pixels 150 in the example of FIG. 1 has 4 rows and 7 columns; however, in general, the array of pixels 150 may have any number of rows and any number of columns.

Each pixel 150 may be configured to detect radiation from a radiation source (not shown) incident thereon and may be configured to measure a characteristic (e.g., the energy of the particles, the wavelength, and the frequency) of the radiation. A radiation may include particles such as photons and subatomic particles. Each pixel 150 may be configured to count numbers of particles of radiation incident thereon whose energy falls in a plurality of bins of energy, within a period of time. All the pixels 150 may be configured to count the numbers of particles of radiation incident thereon within a plurality of bins of energy within the same period of time. When the incident particles of radiation have similar energy, the pixels 150 may be simply configured to count numbers of particles of radiation incident thereon within a period of time, without measuring the energy of the individual particles of radiation.

Each pixel 150 may have its own analog-to-digital converter (ADC) configured to digitize an analog signal representing the energy of an incident particle of radiation into a digital signal, or to digitize an analog signal representing the total energy of a plurality of incident particles of radiation into a digital signal. The pixels 150 may be configured to operate in parallel. For example, when one pixel 150 measures an incident particle of radiation, another pixel 150 may be waiting for a particle of radiation to arrive. The pixels 150 may not have to be individually addressable.

The radiation detector 100 described here may have applications such as in an X-ray telescope, X-ray mammography, industrial X-ray defect detection, X-ray microscopy or microradiography, X-ray casting inspection, X-ray non-destructive testing, X-ray weld inspection, X-ray digital subtraction angiography, etc. It may be suitable to use this radiation detector 100 in place of a photographic plate, a photographic film, a PSP plate, an X-ray image intensifier, a scintillator, or another semiconductor X-ray detector.

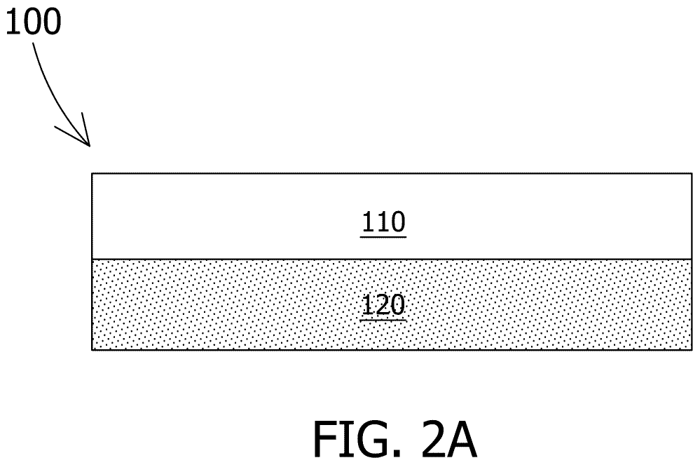

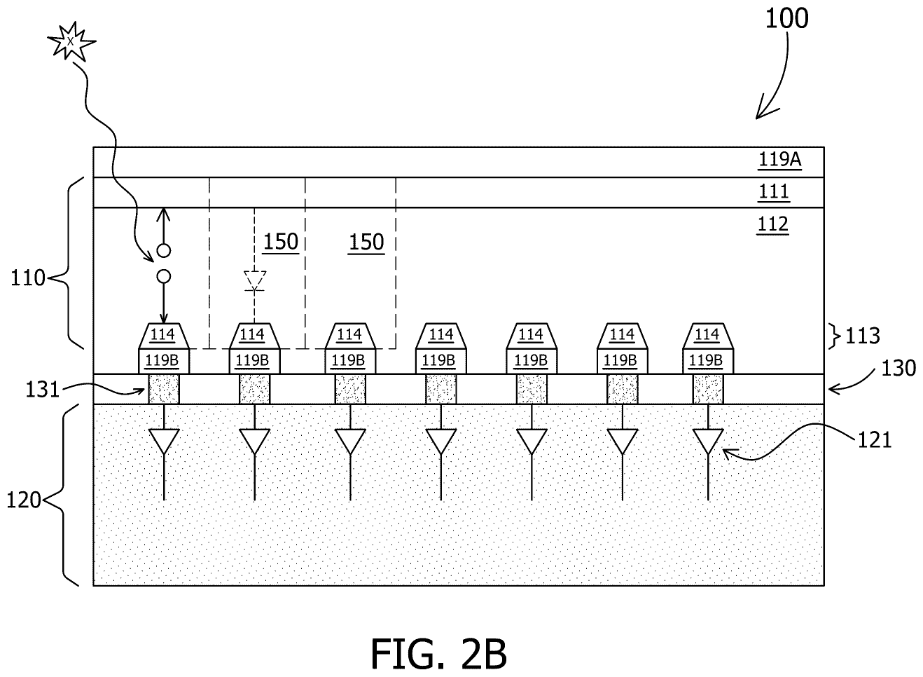

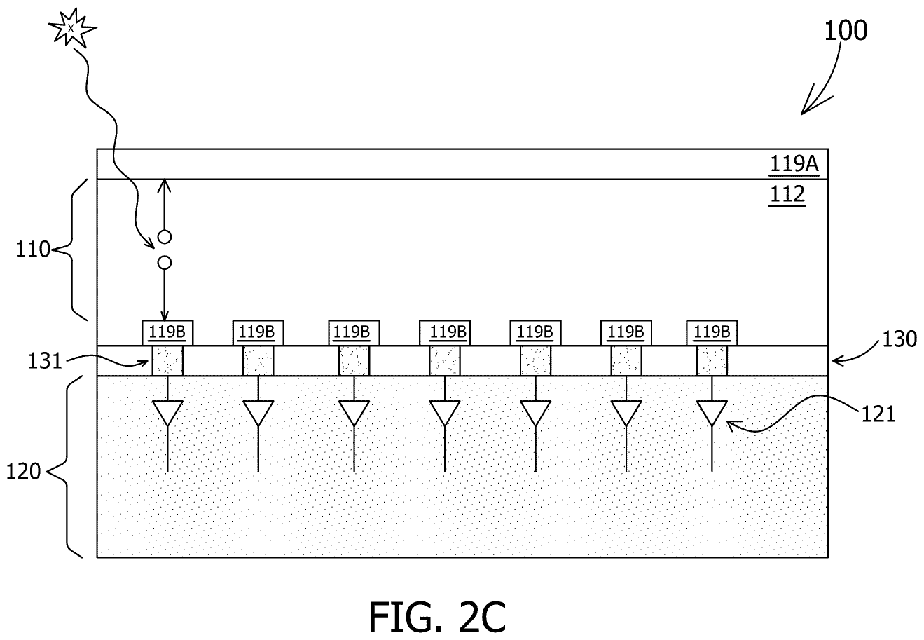

FIG. 2A schematically shows a simplified cross-sectional view of the radiation detector 100 of FIG. 1 along a line 2A-2A, according to an embodiment. Specifically, the radiation detector 100 may include a radiation absorption layer 110 and an electronics layer 120 (e.g., one or more ASICs or application-specific integrated circuits) for processing or analyzing electrical signals which incident radiation generates in the radiation absorption layer 110. The radiation detector 100 may or may not include a scintillator (not shown). The radiation absorption layer 110 may include a semiconductor material such as silicon, germanium, GaAs, CdTe, CdZnTe, or a combination thereof. The semiconductor material may have a high mass attenuation coefficient for the radiation of interest.

FIG. 2B schematically shows a detailed cross-sectional view of the radiation detector 100 of FIG. 1 along the line 2A-2A, as an example. Specifically, the radiation absorption layer 110 may include one or more diodes (e.g., p-i-n or p-n) formed by a first doped region 111, one or more discrete regions 114 of a second doped region 113. The second doped region 113 may be separated from the first doped region 111 by an optional intrinsic region 112. The discrete regions 114 may be separated from one another by the first doped region 111 or the intrinsic region 112. The first doped region 111 and the second doped region 113 may have opposite types of doping (e.g., region 111 is p-type and region 113 is n-type, or region 111 is n-type and region 113 is p-type). In the example of FIG. 2B, each of the discrete regions 114 of the second doped region 113 forms a diode with the first doped region 111 and the optional intrinsic region 112. Namely, in the example in FIG. 2B, the radiation absorption layer 110 has a plurality of diodes (more specifically, 7 diodes corresponding to 7 pixels 150 of one row in the array of FIG. 1, of which only 2 pixels 150 are labeled in FIG. 2B for simplicity). The plurality of diodes may have an electrode 119A as a shared (common) electrode. The first doped region 111 may also have discrete portions.

The electronics layer 120 may include an electronic system 121 suitable for processing or interpreting signals generated by the radiation incident on the radiation absorption layer 110. The electronic system 121 may include an analog circuitry such as a filter network, amplifiers, integrators, and comparators, or a digital circuitry such as a microprocessor, and memory. The electronic system 121 may include one or more ADCs (analog to digital converters). The electronic system 121 may include components shared by the pixels 150 or components dedicated to a single pixel 150. For example, the electronic system 121 may include an amplifier dedicated to each pixel 150 and a microprocessor shared among all the pixels 150. The electronic system 121 may be electrically connected to the pixels 150 by vias 131. Space among the vias may be filled with a filler material 130, which may increase the mechanical stability of the connection of the electronics layer 120 to the radiation absorption layer 110. Other bonding techniques are possible to connect the electronic system 121 to the pixels 150 without using the vias 131.

When radiation from the radiation source (not shown) hits the radiation absorption layer 110 including diodes, particles of the radiation may be absorbed and generate one or more charge carriers (e.g., electrons, holes) by a number of mechanisms. The charge carriers may drift to the electrodes of one of the diodes under an electric field. The electric field may be an external electric field. The electrical contact 119B may include discrete portions each of which is in electrical contact with the discrete regions 114. The term “electrical contact” may be used interchangeably with the word “electrode.” In an embodiment, the charge carriers may drift in directions such that the charge carriers generated by a single particle of the radiation are not substantially shared by two different discrete regions 114 (“not substantially shared” here means less than 2%, less than 0.5%, less than 0.1%, or less than 0.01% of these charge carriers flow to a different one of the discrete regions 114 than the rest of the charge carriers). Charge carriers generated by a particle of the radiation incident around the footprint of one of these discrete regions 114 are not substantially shared with another of these discrete regions 114. A pixel 150 associated with a discrete region 114 may be an area around the discrete region 114 in which substantially all (more than 98%, more than 99.5%, more than 99.9%, or more than 99.99% of) charge carriers generated by a particle of the radiation incident therein flow to the discrete region 114. Namely, less than 2%, less than 1%, less than 0.1%, or less than 0.01% of these charge carriers flow beyond the pixel 150.

FIG. 2C schematically shows a detailed cross-sectional view of the radiation detector 100 of FIG. 1 along the line 2A-2A, according to an alternative embodiment. More specifically, the radiation absorption layer 110 may include a resistor of a semiconductor material such as silicon, germanium, GaAs, CdTe, CdZnTe, or a combination thereof, but does not include a diode. The semiconductor material may have a high mass attenuation coefficient for the radiation of interest. In an embodiment, the electronics layer 120 of FIG. 2C is similar to the electronics layer 120 of FIG. 2B in terms of structure and function.

When the radiation hits the radiation absorption layer 110 including the resistor but not diodes, it may be absorbed and generate one or more charge carriers by a number of mechanisms. A particle of the radiation may generate 10 to 100,000 charge carriers. The charge carriers may drift to the electrical contacts 119A and 119B under an electric field. The electric field may be an external electric field. The electrical contact 119B may include discrete portions. In an embodiment, the charge carriers may drift in directions such that the charge carriers generated by a single particle of the radiation are not substantially shared by two different discrete portions of the electrical contact 119B (“not substantially shared” here means less than 2%, less than 0.5%, less than 0.1%, or less than 0.01% of these charge carriers flow to a different one of the discrete portions than the rest of the charge carriers). Charge carriers generated by a particle of the radiation incident around the footprint of one of these discrete portions of the electrical contact 119B are not substantially shared with another of these discrete portions of the electrical contact 119B. A pixel 150 associated with a discrete portion of the electrical contact 119B may be an area around the discrete portion in which substantially all (more than 98%, more than 99.5%, more than 99.9% or more than 99.99% of) charge carriers generated by a particle of the radiation incident therein flow to the discrete portion of the electrical contact 119B. Namely, less than 2%, less than 0.5%, less than 0.1%, or less than 0.01% of these charge carriers flow beyond the pixel associated with the one discrete portion of the electrical contact 119B.

Radiation Detector Package

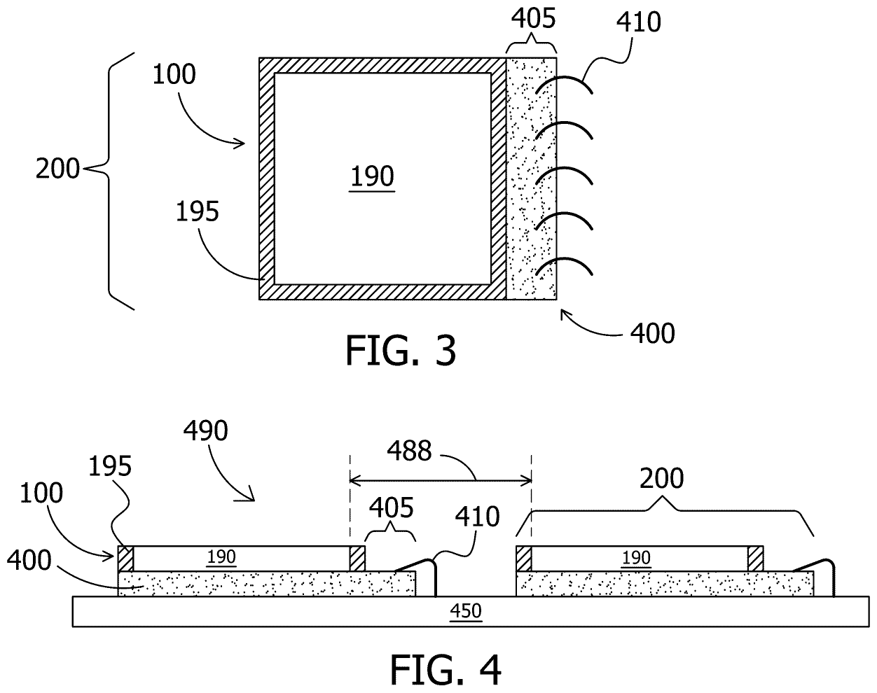

FIG. 3 schematically shows a top view of a package 200 including the radiation detector 100 and a printed circuit board (PCB) 400. The term “PCB” as used herein is not limited to a particular material. For example, a PCB may include a semiconductor. The radiation detector 100 may be mounted to the PCB 400. The wiring between the radiation detector 100 and the PCB 400 is not shown for the sake of clarity. The PCB 400 may have one or more radiation detectors 100. The PCB 400 may have an area 405 not covered by the radiation detector 100 (e.g., for accommodating bonding wires 410). The radiation detector 100 may have an active area 190 which is where the pixels 150 (FIG. 1) are located. The radiation detector 100 may have a perimeter zone 195 near the edges of the radiation detector 100. The perimeter zone 195 has no pixels 150, and the radiation detector 100 does not detect particles of radiation incident on the perimeter zone 195.

Image Sensor

FIG. 4 schematically shows a cross-sectional view of an image sensor 490, according to an embodiment. The image sensor 490 may include a plurality of the packages 200 of FIG. 3 mounted to a system PCB 450. FIG. 4 shows only 2 packages 200 as an example. The electrical connection between the PCBs 400 and the system PCB 450 may be made by bonding wires 410. In order to accommodate the bonding wires 410 on the PCB 400, the PCB 400 may have the area 405 not covered by the radiation detector 100. In order to accommodate the bonding wires 410 on the system PCB 450, the packages 200 may have gaps in between. The gaps may be approximately 1 mm or more. Particles of radiation incident on the perimeter zones 195, on the area 405, or on the gaps cannot be detected by the packages 200 on the system PCB 450. A dead zone of a radiation detector (e.g., the radiation detector 100) is the area of the radiation-receiving surface of the radiation detector, on which incident particles of radiation cannot be detected by the radiation detector. A dead zone of a package (e.g., package 200) is the area of the radiation-receiving surface of the package, on which incident particles of radiation cannot be detected by the radiation detector or detectors in the package. In this example shown in FIG. 3 and FIG. 4, the dead zone of the package 200 includes the perimeter zones 195 and the area 405. A dead zone (e.g., 488) of an image sensor (e.g., image sensor 490) with a group of packages (e.g., packages 200 mounted on the same PCB and arranged in the same layer or in different layers) includes the combination of the dead zones of the packages in the group and the gaps between the packages.

The image sensor 490 including the radiation detectors 100 may have the dead zone 488 incapable of detecting incident radiation. However, the image sensor 490 may capture multiple partial images of an object or scene (not shown), and then these captured partial images may be stitched to form an image of the entire object or scene.

Imaging System—Initial Arrangement

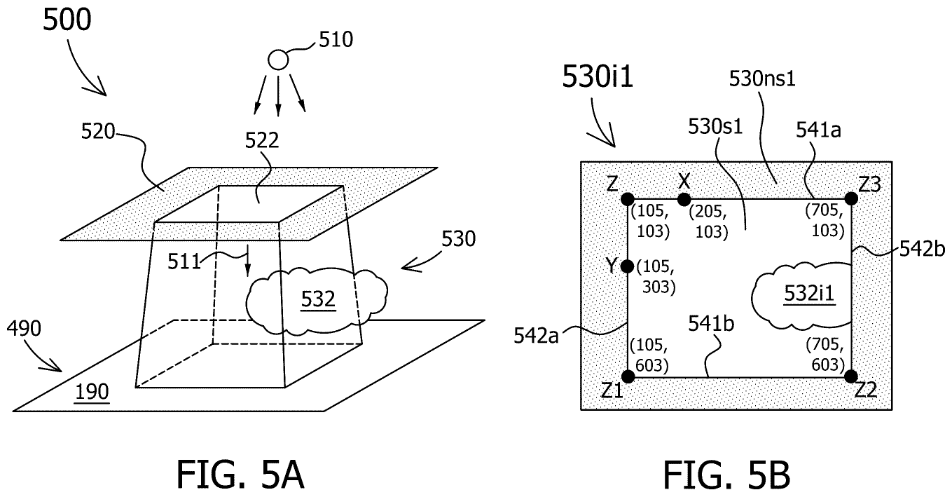

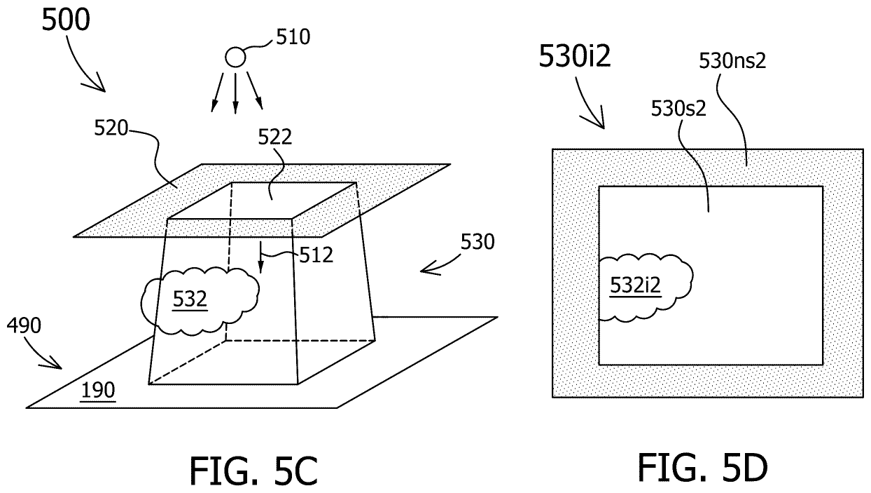

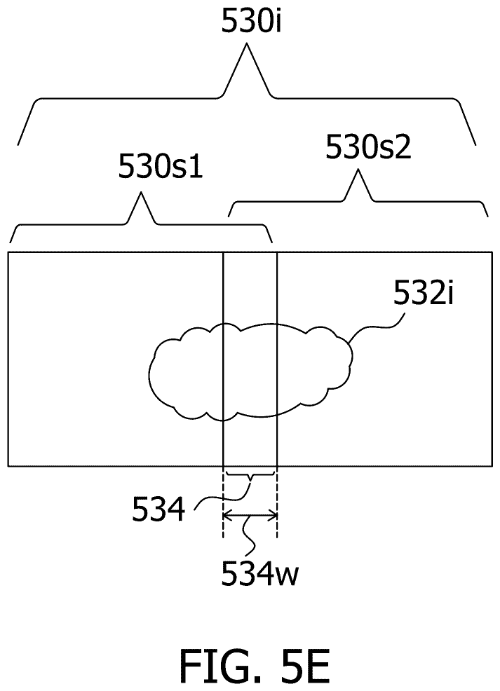

FIG. 5A schematically shows a perspective view of an imaging system 500, according to an embodiment. In an embodiment, the imaging system 500 may include a radiation source 510, a mask 520, and the image sensor 490. The mask 520 may include a mask window 522. For the image sensor 490, only an active area 190 of the image sensor 490 is shown for simplicity. In an embodiment, an object 532 may be positioned in a scene 530 between the mask 520 and the image sensor 490.

In an embodiment, the radiation source 510 may generate radiation toward the mask 520. The portion of the radiation from the radiation source 510 incident on the mask window 522 of the mask 520 may be allowed to pass through the mask 520 (for example, the mask window 522 may be transparent or not opaque), while the portion of the radiation from the radiation source 510 incident on other parts of the mask 520 may be blocked. As a result, after passing through the mask window 522 of the mask 520, the radiation from the radiation source 510 incident on the mask 520 becomes a radiation beam represented by an arrow 511 (hence hereafter this radiation beam may be referred to as the radiation beam 511).

In an embodiment, the mask window 522 of the mask 520 may have a rectangular shape as shown in FIG. 5A. As a result, the radiation beam 511 has the shape of a truncated pyramid as shown in FIG. 5A. In an embodiment, the radiation source 510, the mask 520, and the image sensor 490 may be in a first system arrangement as shown in FIG. 5A. In an embodiment, the relative position of the image sensor 490 with respect to the mask 520 may be such that a plane intersecting all pixels 150 of the active area 190 may be parallel to a surface (facing the radiation source 510) of the mask 520.

First Image Capture

In an embodiment, an imaging session using the image sensor 490 to image the scene 530 (including the object 532) may start with a first image capture as follows. Specifically, in an embodiment, while the radiation source 510, the mask 520, and the image sensor 490 are in the first system arrangement as shown in FIG. 5A, radiation of the radiation beam 511 after passing through the scene 530 may be incident on the active area 190. Using this incident radiation of the radiation beam 511, the active area 190 of the image sensor 490 may capture a first partial image 530i1 (FIG. 5B) of the scene 530 which includes a first partial image 532i1 of the object 532.

With reference to FIG. 5B, the first partial image 530i1 of the scene 530 may include (A) a signal area 530s1 which may include, in an embodiment, picture elements corresponding to the pixels 150 of the active area 190 which receive incident radiation of the radiation beam 511 (in other words, the signal area 530s1 is an image of the radiation beam 511), and (B) a non-signal region 530ns1 which may include, in an embodiment, picture elements corresponding to the pixels 150 of the active area 190 which do not receive incident radiation of the radiation beam 511.

Second Image Capture

In an embodiment, after the active area 190 captures the first partial image 530i1 of the scene 530, the mask 520 and the image sensor 490 may be moved to the right with respect to the scene 530 to a second system arrangement as shown in FIG. 5C such that radiation of a radiation beam 512 from the mask window 522 after passing through the scene 530 may be incident on the active area 190 as shown in FIG. 5C. Then, a second image capture may start as follows. Using this incident radiation of the radiation beam 512, the active area 190 of the image sensor 490 may capture a second partial image 530i2 (FIG. 5D) of the scene 530 which includes a second partial image 532i2 of the object 532. The radiation beam 512 may be generated in a manner similar to the manner in which the radiation beam 511 is created.

With reference to FIG. 5D, the second partial image 530i2 of the scene 530 may include (A) a signal area 530s2 which may include, in an embodiment, picture elements corresponding to the pixels 150 of the active area 190 which receive incident radiation of the radiation beam 512 (in other words, the signal area 530s2 is an image of the radiation beam 512), and (B) a non-signal region 530ns2 which may include, in an embodiment, picture elements corresponding to the pixels 150 of the active area 190 which do not receive incident radiation of the radiation beam 512.

Determination of Signal Areas

In an embodiment, with reference back to FIG. 5A and FIG. 5B, after the active area 190 captures the first partial image 530i1 of the scene 530, the image sensor 490 may determine the signal area 530s1 of the first partial image 530i1.

In an embodiment, the positions and orientations of the mask window 522 and the active area 190 in the first system arrangement may be such that (A) the 2 horizontal border line segments 541a and 541b of the signal area 530s1 are parallel to the rows of picture elements of the partial image 530i1, and (B) the 2 vertical border line segments 542a and 542b of the signal area 530s1 are parallel to the columns of picture elements of the partial image 530i1. In addition, in an embodiment, the lengths (in terms of picture elements) of the border line segments 541a, 541b, 542a, and 542b may be pre-determined (e.g., determined during calibration of the imaging system 500).

As a result, in an embodiment, the image sensor 490 may determine the signal area 530s1 of the first partial image 530i1 as follows. Firstly, in an embodiment, the image sensor 490 may determine a picture element X on the upper horizontal border line segment 541a of the signal area 530s1 by analyzing the signal values of the picture elements of a column of picture elements that intersects the upper horizontal border line segment 541a. Going up in that column of picture elements, the signal values should drop to zero when crossing the upper horizontal border line segment 541a. Therefore, in an embodiment, the first picture element whose signal value is zero when going up in that column of picture elements near the upper horizontal border line segment 541a may be chosen by the image sensor 490 to be the picture element X.

Next, in an embodiment, the image sensor 490 may determine a picture element Y on the left vertical border line segment 542a of the signal area 530s1 in a similar manner, that is, by analyzing the signal values of the picture elements of a row of picture elements that intersects the left vertical border line segment 542a. Going to the left in that row of picture elements, the signal values should drop to zero when crossing the left vertical border line segment 542a. Therefore, in an embodiment, the first picture element whose signal value is zero when going to the left in that row of picture elements near the left vertical border line segment 542a may be chosen by the image sensor 490 to be the picture element Y.

Next, in an embodiment, the image sensor 490 may determine a picture element Z at the upper left corner of the signal area 530s1. In an embodiment, assuming the 2 conditions (A) and (B) mentioned above regarding the positions and orientations of the mask window 522 and the active area 190 are met, the image sensor 490 may choose the picture element on the same row as the picture element X and on the same column as the picture element Y to be the picture element Z.

Next, in an embodiment, the image sensor 490 may determine 3 picture elements Z1, Z2, and Z3 at the three other corners of the rectangular signal area border line 541a,541b,542a,542b of the signal area 530s1 based on the fact that the 2 conditions (A) and (B) mentioned above regarding the positions and orientations of the mask window 522 and the active area 190 are met and on the fact that the lengths (in terms of picture elements) of the border line segments 541a, 541b, 542a, and 542b are pre-determined.

Next, in an embodiment, with the 4 picture elements Z, Z1, Z2, and Z3 at 4 corners of the rectangular signal area 530s1 determined, the image sensor 490 may determine all the picture elements of the signal area 530s1.

For example, assume that the picture element (205,103) is chosen to be the picture element X, and that the picture element (105, 303) is chosen to be the picture element Y (assuming the picture element at the upper left corner of the partial image 530i1 is picture element (1,1)). Then, the picture element (105,103) may be chosen to be the picture elements Z. Assume further that the lengths of the border line segments 541a, 541b, 542a, and 542b are pre-determined to be 600, 600, 500, and 500 picture elements respectively. Then, the 3 other corner picture elements Z1, Z2, and Z3 of the signal area 530s1 are the picture elements (105,603), (705,603), and (705,103), respectively. As a result, the signal area 530s1 includes the picture elements (i,j), i=105, 106, . . . , 704, 705, and j=103, 104, . . . , 602, 603.

In an embodiment, with reference to FIG. 5C and FIG. 5D, after the active area 190 of the image sensor 490 captures the second partial image 530i2 of the scene 530, the image sensor 490 may determine the signal area 530s2 of the partial image 530i2 in a manner similar to the manner in which the image sensor 490 determines the signal area 530s1 of the partial image 530i1 (FIG. 5B).

Alignment of Signal Areas

In an embodiment, with reference to FIG. 5E, after the image sensor 490 determines the 2 signal areas 530s1 and 530s2 as described above, the image sensor 490 may align the 2 signal areas 530s1 and 530s2 resulting in a more complete image 530i of the scene 530 (as shown in FIG. 5E) which includes a more complete image 532i of the object 532.

In an embodiment, the alignment of the signal areas 530s1 and 530s2 may be based on the relative positions of the radiation beams 511 and 512 (FIG. 5A and FIG. 5C) with respect to each other. Specifically, in an embodiment, the relative positions of the radiation beams 511 and 512 with respect to each other may be such that a width 534w of an overlapping region 534 of the 2 signal areas 530s1 and 530s2 is a pre-determined number of picture elements. For example, assume that the relative positions of the radiation beams 511 and 512 with respect to each other are to be such that the width 534w of the overlapping region 534 of the 2 signal areas 530s1 and 530s2 is 198 picture elements, then the 2 signal areas 530s1 and 530s2 can be aligned such that the width 534w of the overlapping region 534 of the 2 signal areas 530s1 and 530s2 is 198 picture elements.

In an embodiment, the picture elements of the signal area 530s1 in the overlapping region 534 may be used in the overlapping region 534 of the stitched image 530i, whereas the picture elements of the signal area 530s2 in the overlapping region 534 may be ignored (i.e., not used in the overlapping region 534 of the stitched image 530i).

Generalization

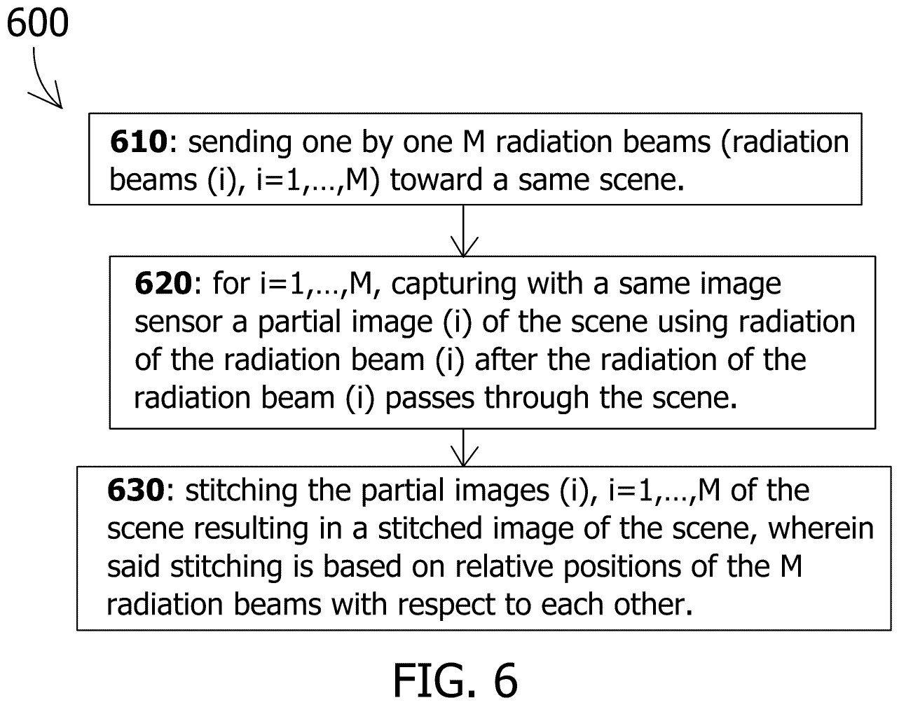

FIG. 6 shows a flowchart 600 generalizing the imaging session described above, according to an embodiment. In step 610, M radiation beams may be sent one by one toward a same scene. For example, M=2 radiation beams 511 and 512 (FIG. 5A and FIG. 5C, respectively) are sent one by one toward the scene 530.

In step 620, for i=1, . . . , M, a partial image (i) of the scene may be captured with a same image sensor using radiation of the radiation beam (i) after the radiation of the radiation beam (i) passes through the scene. For example, the first partial image 530i1 of the scene 530 is captured with the image sensor 490 using radiation of the first radiation beam 511 after the radiation of the first radiation beam 511 passes through the scene 530. Later, the second partial image 530i2 of the scene 530 is captured with the image sensor 490 using radiation of the second radiation beam 512 after the radiation of the second radiation beam 512 passes through the scene 530.

In step 630, the partial images (i), i=1, . . . , M of the scene may be stitched resulting in a stitched image of the scene, wherein said stitching is based on relative positions of the M radiation beams with respect to each other. For example, the partial images 530i1 and 530i2 of the scene 530 are stitched (i.e., their signal areas 530s1 and 530s2 are determined and then aligned) resulting in the stitched image 530i of the scene 530, wherein said stitching is based on the relative positions of the 2 radiation beams 511 and 512 with respect to each other. Here, stitching multiple partial images of the scene 530 includes determining their signal areas and then aligning the determined signal areas to form a stitched image of the scene 530.

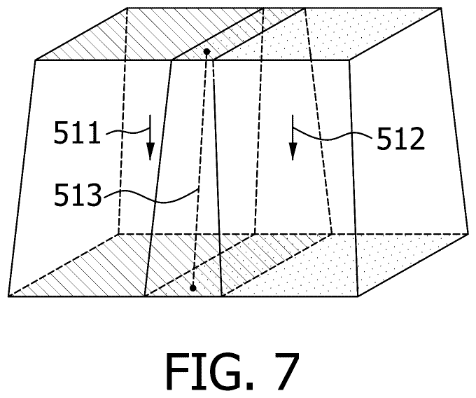

FIG. 7 shows the 2 radiation beams 511 and 512 side by side. In the embodiments described above, the 2 radiation beams 511 and 512 share some radiation particle paths (e.g., radiation particle path 513). A radiation beam has a radiation particle path if at least a radiation particle of that radiation beam follows (or propagates along) that radiation particle path. In an alternative embodiment, the 2 radiation beams 511 and 512 do not share any radiation particle path. In this alternative embodiment, the 2 signal areas 530s1 and 530s2 of the 2 partial images 530i1 and 530i2 of the scene 530 do not overlap. However, in this alternative embodiment, the 2 signal areas 530s1 and 530s2 may still be determined and then aligned based on the relative positions of the radiation beams 511 and 512 with respect to each other, but the resulting stitched image 530i has 2 separate regions which are the 2 signal areas 530s1 and 530s2. This alternative embodiment is not shown.

In the embodiments described above, the imaging session uses only 2 radiation beams 511 and 512 one by one to generate 2 partial images 530i1 and 530i2 of the scene 530 respectively. In general, the imaging session may use M radiation beams one by one (M is an integer greater than 1) to generate M partial images of the scene 530. The resulting M partial images of the scene 530 may be stitched (i.e., their signal areas are determined and then aligned) to form a stitched image of the scene 530 based on the relative positions of the M radiation beams with respect to each other. Described in details above is the case where M=2.

In an embodiment, the M radiation beams may overlap such that for i=1, . . . , M−1, the radiation beam (i) and the radiation beam (i+1) share some radiation particle paths.

In an embodiment, with reference to FIG. 5A and FIG. 5C, both the radiation beams 511 and 512 after passing through the scene 530 may fall entirely within the same active area 190 of the image sensor 490 as shown in FIG. 5A and FIG. 5C. In an alternative embodiment, the radiation beam 511 after passing through the scene 530 may fall entirely within the active area 190 as shown in FIG. 5A, but the radiation beam 512 after passing through the scene 530 may fall entirely within another active area 190 of the image sensor 490 (now shown).

In an embodiment, the stitching of the partial images 530i1 and 530i2 as described above is not based on the positions of the image sensor 490 in the first and second system arrangements with respect to each other.

In the embodiments described above, with reference to FIG. 5A— FIG. 5B, the determination of the 4 corner picture elements Z, Z1, Z2, and Z3 is based on the conditions that (A) the 2 horizontal border line segments 541a and 541b of the signal area 530s1 are parallel to the rows of picture elements of the partial image 530i1, and (B) the 2 vertical border line segments 542a and 542b of the signal area 530s1 are parallel to the columns of picture elements of the partial image 530i1. In a general case, even without the conditions (A) and (B) mentioned above being met, the corner picture element Z may be determined by first (A) determining two picture elements X1 and X2 (not shown) on the border line segment 541a, and two picture elements Y1 and Y2 on the border line segment 542a, and then (B) choosing a picture element which is both (i) collinear with X1 and X2 and (ii) collinear with Y1 and Y2 to be the picture element Z. The determination of the picture elements X1, X2, Y1, and Y2 may be similar to the determination of the picture elements X and Y described above. In the general case, the determination of the corner picture elements Z1, Z2, and Z3 may be similar to the determination of the corner picture element Z described immediately above. After the 4 corner picture elements Z, Z1, Z2, and Z3 of the rectangular signal area 530s1 are determined, the rectangular signal area 530s1 itself may be determined.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method, comprising:
sending one by one M radiation beams (radiation beams (i), i=1, . . . , M) toward a same scene, M being an integer greater than 1;
for i=1, . . . , M, capturing with a same image sensor a partial image (i) of the scene using radiation of the radiation beam (i) after the radiation of the radiation beam (i) passes through the scene; and
stitching the partial images (i), i=1, . . . , M of the scene resulting in a stitched image of the scene, wherein said stitching is based on relative positions of the M radiation beams with respect to each other.

2. The method of claim 1, wherein for i=1, . . . , M−1, the radiation beam (i) and the radiation beam (i+1) share some radiation particle paths.

3. The method of claim 1, wherein for i=1, . . . , M, the radiation beam (i) after passing through the scene falls entirely within an active area of the image sensor.

4. The method of claim 1, wherein for i=1, . . . , M, the radiation beam (i) after passing through the scene falls entirely within a same active area of the image sensor.

5. The method of claim 1,
wherein for i=1, . . . , M, the image sensor is at a position (i) with respect to the scene when the image sensor captures the partial image (i) of the scene, and
wherein said stitching is not based on the positions (i), i=1, . . . , M of the image sensor with respect to each other.

6. The method of claim 1, wherein said stitching the partial images (i), i=1, . . . , M comprises:
for i=1, . . . , M, determining a signal area (i) of the partial image (i); and
aligning the signal areas (i), i=1, . . . , M resulting in the stitched image of the scene, wherein said aligning is based on the relative positions of the M radiation beams with respect to each other.

7. The method of claim 6, wherein said determining the signal area (i) comprises determining multiple picture elements of the partial image (i) on a signal area border line (i) of the signal area (i).

8. The method of claim 7, wherein the signal area border line (i) has a shape of a rectangle.

9. The method of claim 1,
wherein said sending one by one the M radiation beams comprises moving a mask between a radiation source and the scene as the image sensor captures the partial images (i), i=1, . . . , M, and
wherein the mask comprises a mask window such that radiation from the radiation source that passes through the mask window results in the M radiation beams.

10. The method of claim 1, wherein said capturing with the same image sensor comprises moving the image sensor with respect to the scene as the image sensor captures the partial images (i), i=1, . . . , M.

11. An imaging system, comprising:
a radiation beam generator configured to generate one by one M radiation beams (radiation beams (i), i=1, . . . , M) toward a same scene, M being an integer greater than 1; and
an image sensor configured to (A) for (i), i=1, . . . , M, capture a partial image (i) of the scene using radiation of the radiation beam (i) after the radiation of the radiation beam (i) passes through the scene, and (B) stitch the partial images (i), i=1, . . . , M of the scene resulting in a stitched image of the scene based on relative positions of the M radiation beams with respect to each other.

12. The imaging system of claim 11, wherein for i=1, . . . , M−1, the radiation beam (i) and the radiation beam (i+1) share some radiation particle paths.

13. The imaging system of claim 11, wherein for i=1, . . . , M, the radiation beam (i) after passing through the scene falls entirely within an active area of the image sensor.

14. The imaging system of claim 11, wherein for i=1, . . . , M, the radiation beam (i) after passing through the scene falls entirely within a same active area of the image sensor.

15. The imaging system of claim 11,
wherein for i=1, . . . , M, the image sensor is configured to be at a position (i) with respect to the scene when the image sensor captures the partial image (i) of the scene, and
wherein said stitching of the partial images (i), i=1, . . . , M is not based on the positions (i), i=1, . . . , M of the image sensor with respect to each other.

16. The imaging system of claim 11, wherein the image sensor is configured to stitch the partial images (i), i=1, . . . , M of the scene by:
for i=1, . . . , M, determining a signal area (i) of the partial image (i), and
aligning the signal areas (i), i=1, . . . , M resulting in the stitched image of the scene, wherein said aligning is based on the relative positions of the M radiation beams with respect to each other.

17. The imaging system of claim 16, wherein said determining the signal area (i) comprises determining multiple picture elements of the partial image (i) on a signal area border line (i) of the signal area (i).

18. The imaging system of claim 17, wherein the signal area border line (i) has a shape of a rectangle.

19. The imaging system of claim 11,
wherein the radiation beam generator comprises a radiation source and a mask which comprises a mask window, and
wherein the mask is configured to move and allow some radiation from the radiation source to pass through the mask window resulting in the radiation beams (i), i=1, . . . , M.

20. The imaging system of claim 11, wherein the image sensor is configured to move with respect to the scene as the image sensor captures the partial images (i), i=1, . . . , M.
